(19) United States
(12) Patent Application Publication
(10) Pub. No.: US 2025/0046472 A1
(43) Pub. Date: Feb. 6, 2025

(54) SYSTEM AND METHOD FOR DISEASE PREDICTION

(71) Applicant: Siemens Healthcare Diagnostics Inc., Tarrytown, NY (US)

(72) Inventors: Kratika Gupta, Bangalore (IN); Nivedita Mitra, Bangalore (IN); Fernando Chaves, New York, NY (US); Graham Gibbs, Taunton (GB); Val Jones, Dublin (IE)

(21) Appl. No.: 18/837,457

(22) PCT Filed: Feb. 23, 2023
(86) PCT No.: PCT/US2023/063091

§ 371 Date: Aug. 9, 2024

Related U.S. Application Data
(60) Provisional application No. 63/268,435, filed on Feb. 24, 2022.

(30) Foreign Application Priority Data
Feb. 24, 2022 (IN) 202231010035
Jan. 12, 2023 (IN) 202331002562

(51) Int. Cl. G16H 50/80; G16H 10/40; G16H 50/20

(52) U.S. Cl. CPC G16H 50/80; G16H 10/40; G16H 50/20

(57) ABSTRACT

A system and a method for predicting presence of a disease in a subject are disclosed. In one aspect, the system includes one or more processors, a medical database coupled to the one or more processors, and a disease prediction module. The disease prediction module is configured to: receive a plurality of parameters associated with the subject; determine a threshold for sensitivity and/or specificity associated with a trained machine learning model; and predict, using the trained machine learning model, the presence of the disease in the subject based on the desired sensitivity and specificity and the plurality of parameters associated with the subject and output the prediction on an output unit.

### CROSS REFERENCE TO RELATED APPLICATIONS

This application is a § 371 nationalization of PCT Application Serial No. PCT/US2023/063091, filed Feb. 23, 2023, designating the United States, and this application also claims priority or the benefit under 35 U.S.C. § 119 of U.S. Provisional Patent Application No. 63/268,435 filed Feb. 24, 2022, Indian Patent Application number 202231010035 filed Feb. 24, 2022, and Indian Patent Application number 202331002562 filed Jan. 12, 2023, all of which are herein entirely incorporated by reference.

### TECHNICAL FIELD

The present disclosure relates to the fields of medicine, health of a subject, analysis of a sample, and more particularly to the field of prediction of a presence of disease in a subject.

### BACKGROUND

Prediction of disease is important not just in providing a timely treatment to the patients, but also in managing resources and logistics involved in the disease management process. For example, the pandemic arising due to SARS-COV-2 virus increased the burden on healthcare systems across the world. An accelerated growth in the number of individuals required to undergo testing for detection of SARS-COV-2 virus resulted in shortage of infectious disease test kits such as reverse transcriptase polymerase chain reaction (RT-PCR) test kits, delay in providing test results to individuals, and delay in providing timely medical support to patients when presence of SARS-COV-2 virus was not suspected. Prediction of presence of one or more pathogens such as a virus, (e.g., SARS-COV-2), bacteria, or the like in patients may be important in such scenarios.

Currently, there are no reliable ways through which presence of a pathogen such as a virus, (e.g., SARS-COV-2 virus), in a subject may be predicted efficiently, without an RT-PCR test or an antigen test. There is a continuing need for improved systems and methods of predicting the presence of a pathogen such as a virus or bacteria in a subject, and/or detecting an increase in infection rates in a population to improve the utilization of medical resources.

### SUMMARY

A system for predicting a presence of a disease in a subject is disclosed. In one aspect of the present disclosure, the system includes a processor and a memory. Additionally, the memory includes a disease prediction module configured to receive a plurality of parameters associated with the subject and determine a threshold for sensitivity and/or specificity associated with a trained machine learning model. Additionally, the module is configured to predict using the trained machine learning model the presence of the disease in the subject and the plurality of parameters associated with the subject and output the prediction on an output unit.

In another aspect of the present disclosure, a method for predicting a presence of a disease in a subject is disclosed. The method includes receiving a plurality of parameters associated with the subject. Additionally, the method includes determining a threshold for sensitivity and/or specificity associated with a trained machine learning model. Furthermore, the method includes predicting, using a trained machine learning model, the presence of the disease in the subject based on the sensitivity and specificity of the disease and the plurality of parameters associated with the subject. Further, the method includes outputting the prediction on an output unit.

In embodiments, the present disclosure includes an article of manufacture, (e.g., a system or a component thereof), including a non-transitory computer-readable medium with instructions encoded thereon, the instructions configured to cause one or more processors to perform a method including: predicting a presence of a disease in accordance with the present disclosure.

In embodiments, the present disclosure includes non-transient computer readable medium with instructions encoded thereon, the instructions configured to cause one or more processors to perform a method including: predicting a presence of a disease in accordance with the present disclosure.

In embodiments, the present disclosure includes predicting a presence of a disease in a subject in accordance with the present disclosure and subsequently treating the subject in need thereof.

In embodiments, the present disclosure includes a system for predicting a presence of a disease in a population. In one aspect of the present disclosure, the system includes a processor and a memory. Additionally, the memory includes a disease prediction module configured to receive a plurality of parameters associated with one or more subjects and determine a threshold for sensitivity and/or specificity associated with a trained machine learning model. Additionally, the module is configured to predict using the trained machine learning model the presence of the disease in one or more subjects or a population of subjects and the plurality of parameters associated with the subject or population of subjects and output the prediction on an output unit.

This summary is provided to introduce a selection of concepts in a simplified form further described below in the following description. It is not intended to identify features or essential features of the claimed subject matter. Furthermore, the claimed subject matter is not limited to implementations that solve any or all disadvantages noted in any part of this disclosure.

### BRIEF DESCRIPTION OF THE DRAWINGS

The present disclosure is further described hereinafter with reference to illustrated embodiments shown in the accompanying drawings, in which:

FIG. 1 illustrates a system for predicting presence of a disease in a subject, according to an embodiment.

FIG. 2 illustrates a process flow for predicting presence of a disease in a subject, according to an embodiment of the present disclosure.

FIG. 3 illustrates a receiver operating characteristic (ROC) curve for choosing threshold for sensitivity and specificity associated with a trained machine learning model for a requirement scenario, according to an embodiment of the present disclosure.

FIG. 4 depicts a ROC curve of a 48-parameter ensemble model computed using a test set of the present disclosure.

FIG. 5 depicts model interpretability: features arranged in decreasing order of importance as computed by the MIMIC LightGBM explainer.

FIGS. 6A, 6B, and 6C depict use case embodiments such as in a pandemic scenario, and a use case for an endemic scenario at different regions (1, 2, and 3) of the ROC curve.

### DETAILED DESCRIPTION

Hereinafter, systems, methods, and articles of manufacture for carrying out embodiments of the present disclosure are described in detail. The various embodiments are described with reference to the drawings, wherein like reference numerals are used to refer to like elements throughout. In the following description, for purpose of explanation, numerous specific details are set forth to provide a thorough understanding of one or more embodiments. It may be evident that such embodiments may be practiced without these specific details. In other instances, well known materials or methods have not been described in detail to avoid unnecessarily obscuring embodiments of the present disclosure. While the disclosure is susceptible to various modifications and alternative forms, specific embodiments thereof are shown by way of example in the drawings and are herein be described in detail. It should be understood, however, that there is no intent to limit the disclosure to the particular forms disclosed, but on the contrary, the disclosure is to cover all modifications, equivalents, and alternatives falling within the spirit and scope of the present disclosure. Disclosed embodiments provide a system and method for predicting disease in a subject.

Advantages of embodiments of the present disclosure include excellent and robust prediction of disease resulting from one or more pathogens to provide, among other things, timely treatment to the patients or subjects in need thereof, and improved management of resources and logistics involved in the disease management process. Embodiments of the present disclosure reduce the burden on healthcare systems across the world, prevent test kit shortages, and shorten the duration on waiting for individual test results, and shorten the waiting period for medical support to patients when presence of a pathogen is not suspected.

COVID-19 testing using RT-PCR (reverse transcriptase-polymerase chain reaction) and antigen tests scaled up massively since the onset of the COVID-19 pandemic, however, there are multiple scenarios where a CBC/Diff based predictive algorithm may be applied in the clinical value chain both in the pandemic and endemic scenarios. First, a CBC/Diff based predictive algorithm may act as a substitute for testing in countries dealing with unavailability or shortages of such tests. Second, quick flagging by a highly sensitive hematology-based machine learning (ML) algorithm (see, e.g., Algorithm A described below, with a threshold of 0.41) may reduce the testing burden in a pandemic scenario. Third, an algorithm with high negative predictive value (NPV) (see, e.g., Algorithm A, with a threshold of 0.33) may reduce the number of patients undergoing testing before procedures like surgery, thereby reducing time to patient care. Fourth, in an endemic scenario, an algorithm with high specificity (see, e.g., Algorithm A with a threshold of 0.67) may be used for flagging asymptomatic individuals who may then be tested to confirm the infection. Accordingly, the same ML algorithm such as Algorithm A with different classification thresholds (such as a threshold value, e.g., 0.4-1.0, 0.4-0.97, 0.4-0.95, 0.4-0.7, 0.4-0.6, 0.4-0.5, 0.4-0.45, 0.80-0.95, or 0.99-1.0) may be used in multiple scenarios described herein.

### Certain Definitions

As utilized in accordance with the present disclosure, the following terms, unless otherwise indicated, shall be understood to have the following meanings. Unless otherwise clear from context, (i) the term “a” may be understood to mean “at least one;” (ii) the terms “comprising” and “including” may be understood to encompass itemized components or acts whether presented by themselves or together with one or more additional components or acts; and (iii) where ranges are provided, endpoints are included.

The use of the term “at least one” is understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100 or 1000 are not to be considered limiting, as higher limits may also produce satisfactory results. In addition, the use of the term “at least one of X, Y, and Z” is understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and understood by persons having ordinary skill in the art. In embodiments, the term “about,” when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that there may be no limit on the number of items or terms in any combination, unless otherwise apparent from the context.

As used herein the terms “CBC/Diff” or “blood cell count with differential” refers to a measure of the number of red blood cells, white blood cells, and platelets in the blood, including the different types of white blood cells (neutrophils, lymphocytes, monocytes, basophils, and eosinophils). In embodiments, the amount of hemoglobin (substance in the blood that carries oxygen) and the hematocrit (the amount of whole blood that is made up of red blood cells) are also measured.

As used herein, “diagnostic test” is an act or series of acts that is or has been performed to attain information that is useful in determining whether a patient has a disease, disorder or condition and/or in classifying a disease, disorder or condition into a phenotypic category or any category having significance with regard to prognosis of a disease, disorder or condition, or likely response to treatment (either treatment in general or any particular treatment) of a disease, disorder or condition. Similarly, “diagnosis” refers to providing any type of diagnostic information, including, but not limited to, whether a subject is likely to have or develop a disease, disorder or condition, state, staging or characteristic of a disease, disorder or condition as manifested in the subject, information related to the nature or classification of a tumor, information related to prognosis and/or information useful in selecting an appropriate treatment or additional diagnostic testing. Selection of treatment may include the choice of a particular therapeutic agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc. Selection of additional diagnostic testing may include more specific testing for a given disease, disorder, or condition.

As used herein, the terms “disease” and “disorder” are used interchangeably to refer to a condition in a subject including a harmful deviation from the normal structural or functional state of an organism. Non-limiting examples of diseases/disorders include a subject having one or more viral infections, one or more bacterial infections, one or more fungal or parasitic infections, or sepsis.

As used herein, the term “viral infection” means the invasion by, multiplication and/or presence of a virus in a cell or a subject. In one embodiment, a viral infection is an “active” infection, i.e., one in which the virus is replicating in a cell or a subject. Such an infection is characterized by the spread of the virus to other cells, tissues, and/or organs, from the cells, tissues, and/or organs initially infected by the virus. An infection may also be a latent infection, i.e., one in which the virus is not replicating.

As used herein, the term “bacterial infection” means the invasion by, multiplication and/or presence of a bacteria in a cell or a subject.

As used herein, the term “fungal infection” means the invasion by, multiplication and/or presence of a fungus in a cell or a subject.

As used herein, the term “pathogen infection” means the invasion by, multiplication and/or presence of a pathogen (such as a bacterial, fungal, parasitic, or viral pathogen) in a cell or a subject.

As used herein, the term “sample” refers to a biological sample obtained or derived from a human subject, as described herein. In some embodiments, a biological sample includes biological tissue or fluid. In some embodiments, a biological sample may include blood; blood cells; tissue or fine needle biopsy samples; cell-containing body fluids; free floating nucleic acids; cerebrospinal fluid; lymph; tissue biopsy specimens; surgical specimens; other body fluids, secretions, and/or excretions; and/or cells therefrom. In some embodiments, a biological sample includes cells obtained from an individual, e.g., from a human or animal subject. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood). In some embodiments, a sample is cardiac tissue obtained from the subject. In some embodiments, as is clear from context, the term “sample” refers to a preparation that is obtained by processing (e.g., by removing one or more components of and/or by adding one or more agents to) a primary sample. For example, filtering using a semi-permeable membrane. As another example of sample processing, the sample may be a plasma sample that is treated with an anticoagulant selected from the group consisting of EDTA, heparin, and citrate. As another example of sample processing, the sample may be processed to isolate one or more proteins (e.g., by capturing proteins with one or more antibodies). A “processed sample” may include, for example, nucleic acids or polypeptides extracted from a sample or obtained by subjecting a primary sample to techniques such as amplification or reverse transcription of mRNA, isolation and/or purification of certain components.

As used herein, the term “SARS COV-2 variant” or “viral variant” or simply “variant” when used in reference to SARS COV-2 (as apparent from context), is a SARS CoV-2 virus that includes one or more genetic changes relative to a SARS COV-2 reference strain or one or more predominant viral variants already circulating in a population. In some embodiments, a SARS COV-2 variant includes one or more genomic mutations in an S gene sequence. In some embodiments, a SARS COV-2 variant includes one or more genomic mutations relative to a reference genome sequence or portion thereof. In some embodiments, a SARS COV-2 variant includes one or more genomic mutations in an S gene sequence relative to an S gene sequence of a reference genome. In some embodiments, a reference genome sequence corresponds to that of the Wuhan-Hu1 strain (the first genetic sequence identified) or USA-WA1/2020 strain (the first identified in the United States) or portion thereof. In some embodiments, a SARS COV-2 variant includes one or more genomic mutations relative to one or more predominant viral variants circulating in a population. In some embodiments, a SARS COV-2 variant includes one or more genomic mutations in an S gene sequence relative to an S gene sequence of a predominant viral variant circulating in a population.

As used herein, the term “subject” refers to an organism, for example, a mammal (e.g., a human). In some embodiments a human subject is an adult, adolescent, or pediatric subject. In some embodiments, a subject is at least 50, at least 55, at least 60, at least 65, at least 70, at least 75, or at least 80 years of age. In some embodiments, a subject is suffering from a disease, disorder, or condition, e.g., a disease, disorder, or condition that may be treated as provided herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered. In some embodiments, a human subject is an adult, adolescent, or pediatric subject. In some embodiments, a subject is at risk for a SARS COV-2 viral infection. In some embodiments, a subject is at risk for a viral infection, a bacterial infection, sepsis, or combinations thereof. In some embodiments, a subject has been exposed or is suspected to have a COVID-19 infection. In some embodiments, a subject is susceptible to COVID-19. In some embodiments, a susceptible subject is predisposed to and/or shows an increased risk (as compared to the average risk observed in a reference subject or population) of developing COVID-19. In some embodiments, a subject displays one or more symptoms of a SARS COV-2 infection. In some embodiments, a subject does not display a particular symptom or characteristic of COVID-19. In some embodiments, a subject does not display any symptom or characteristic of COVID-19 (i.e., is asymptomatic). In some embodiments, a subject is an individual to whom diagnosis and/or therapy is and/or has been administered.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the term “threshold value” refers to a value (or values) that are used as a reference to attain information on and/or classify the results of a measurement, for example, the results of a measurement attained in an assay. A threshold value may be determined based on one or more control samples. A threshold value may be determined prior to, concurrently with, or after the measurement of interest is taken. In some embodiments, a threshold value may be a range of values. In some embodiments, a threshold value may be a value (or range of values) reported in the relevant field (e.g., a value found in a standard table).

The terms “treatment” or “treating” of a subject includes the application or administration of a compound to a subject with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being. In embodiments, the term “treating” means reducing or ameliorating the progression, severity, and/or duration of COVID-19, or ameliorating one or more symptoms of COVID-19 caused by administration of one or more therapies (e.g., one or more therapeutic agents). In a particular embodiment, the term “treatment” means to ameliorate measurable physical parameters of COVID-19. In embodiments, the term “treating” means reducing or ameliorating the progression, severity, and/or duration of a viral, bacterial, or fungal infection or ameliorating one or more symptoms of a viral, bacterial, or fungal infection caused by administration of one or more therapies (e.g., one or more therapeutic agents). In a particular embodiment, the term “treatment” means to ameliorate measurable physical parameters of a viral, bacterial, or fungal infection. In embodiments, the term “treating” means reducing or ameliorating the progression, severity, and/or duration of sepsis, or ameliorating one or more symptoms of a sepsis caused by administration of one or more therapies (e.g., one or more therapeutic agents). In a particular embodiment, the term “treatment” means to ameliorate measurable physical parameters of sepsis. In embodiments, “treating” changes the natural or presenting state of a subject.

As used herein, the term “therapeutically effective amount” means the amount of compound that, when administered to a subject for treating or preventing a particular disorder, disease, or condition, is sufficient to effect such treatment or prevention of that disorder, disease, or condition. Dosages and therapeutically effective amounts may vary for example, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject and the properties of the compounds (e.g., bioavailability, stability, potency, toxicity, etc.), and the particular disorder(s) the subject is suffering from. In addition, the therapeutically effective amount that is administered intravenously may depend on the subject's blood parameters, e.g., lipid profile, insulin levels, glycemia or liver metabolism. The therapeutically effective amount will also vary according to the severity of the disease state, organ function, or underlying disease or complications. Such appropriate doses may be determined using any available assays. When one or more of the compounds or therapeutic agents is to be administered to humans, a physician may for example, prescribe a relatively low dose at first, subsequently increasing the dose until an appropriate response is obtained.

As used herein, the term “viral disease” refers to the pathological state resulting from the presence of a virus in a cell or subject or the invasion of a cell or subject by a virus.

As used herein, the term “influenza virus disease” refers to the pathological state resulting from the presence of an influenza (e.g., influenza A or B virus) virus in a cell or subject or the invasion of a cell or subject by an influenza virus. In specific embodiments, the term refers to a respiratory illness caused by an influenza virus.

As used herein, the term “COVID-19 disease” refers to the pathological state resulting from the presence of SARS-COV-2 (e.g., a SARS-COV-2 variant) virus in a cell or subject or the invasion of a cell or subject by a SARS-COV-2 virus or variant thereof. In specific embodiments, the term refers to a respiratory illness caused by a corona virus such as SARS-COV-2 or variant thereof.

The term “sepsis” has been used to describe a variety of clinical conditions related to systemic manifestations of inflammation accompanied by an infection. Because of clinical similarities to inflammatory responses secondary to non-infectious etiologies, identifying sepsis has been a particularly challenging diagnostic problem. Sepsis definitions may change over time and may also vary across various clinical/hospital systems (e.g., Singer M et al., “The third international consensus definitions for sepsis and septic shock (Sepsis-3),” JAMA. 2016-2-23; 315 (8): 801-10; Bone R C et al., “American College of Chest Physicians/Society of Critical Care Medicine Consensus Conference: definitions for sepsis and organ failure and guidelines for the use of innovative therapies in sepsis,” Crit Care Med. 1992; 20 (6): 864-874; Levy M M et al.; International Sepsis Definitions Conference. 2001 SCCM/ESICM/ACCP/ATS/SIS International Sepsis Definitions Conference. Intensive Care Med. 2003; 29 (4): 530-538. Centers for Disease Control and Prevention. Hospital toolkit for adult sepsis surveillance. 2018. See e.g., www.cdc.gov/sepsis/pdfs/Sepsis-Surveillance-Toolkit-Mar-2018_508.pdf). All such definitions for sepsis, not limited to the examples cited above, are intended for the purposes of the present application.

### DETAILED DESCRIPTION OF CERTAIN EMBODIMENTS

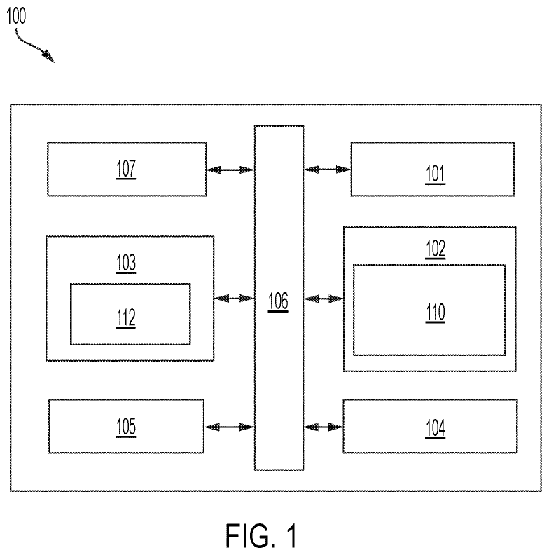

FIG. 1 is a block diagram of system 100 in which an embodiment may be implemented, for example, as a system 100 for predicting presence for a disease, configured to perform the process as described therein. In FIG. 1, the system 100 includes a processor 101, a memory 102, a storage unit 103, an input unit 104, a bus 106, an output unit 105, and a network interface 107.

The processor 101, as used herein, means any type of computational circuit, such as, but not limited to, a microprocessor, microcontroller, complex instruction set computing microprocessor, reduced instruction set computing microprocessor, very long instruction word microprocessor, explicitly parallel instruction computing microprocessor, graphics processor, digital signal processor, or any other type of processing circuit. The processor 101 may also include embedded controllers, such as generic or programmable logic devices or arrays, application specific integrated circuits, single-chip computers, and the like.

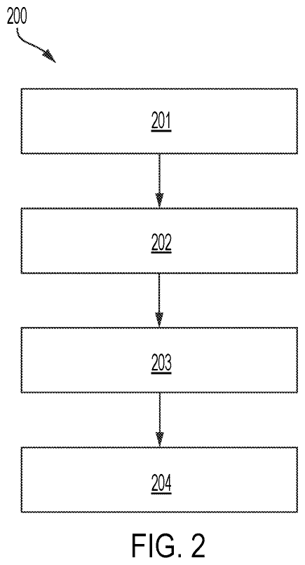

The memory 102 may be volatile memory and non-volatile memory. The memory 102 may be coupled for communication with the processor 101. The processor 101 may execute instructions and/or code stored in the memory 102. A variety of computer-readable storage media may be stored in and accessed from the memory 102. The memory 102 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 102 includes a disease prediction module 110 stored in the form of machine-readable instructions on any of the above-mentioned storage media and may be in communication to and executed by processor 101. When executed by the processor 101, the disease prediction module 110 causes the processor 101 to predict presence of a disease in a subject. Method acts executed by the processor 101 to achieve the abovementioned functionality are elaborated upon in detail in FIG. 2.

The storage unit 103 may be a non-transitory storage medium which stores a medical database 112. The medical database 112 is a repository of patient data, including blood parameters, which is maintained by a healthcare service provider. The input unit 104 may include input means such as keypad, touch-sensitive display, camera (such as a camera receiving gesture-based inputs), etc. capable of receiving in-put signal. The bus 106 acts as interconnect between the processor 101, the memory 102, the storage unit 103, the input unit 104, the output unit 105 and the network interface 107.

Those of ordinary skilled in the art will appreciate that the hardware depicted in FIG. 1 may vary for particular implementations. For example, other peripheral devices such as an optical disk drive and the like, Local Area Network (LAN)/Wide Area Network (WAN)/Wireless (e.g., Wi-Fi) adapter, graphics adapter, disk controller, input/output (I/O) adapter also may be used in addition or in place of the hardware depicted. The depicted example is provided for the purpose of explanation only and is not meant to imply architectural limitations with respect to the present disclosure.

The system 100 in accordance with an embodiment of the present disclosure includes an operating system employing a graphical user interface. The operating system permits multiple display windows to be presented in the graphical user interface simultaneously with each display window providing an interface to a different application or to a different instance of the same application. A cursor in the graphical user interface may be manipulated by a user through a pointing device. The position of the cursor may be changed and/or an event such as clicking a mouse button, generated to actuate a desired response. One of various commercial operating systems, such as a version of Microsoft Windows™, a product of Microsoft Corporation located in Redmond, Washington may be employed if suitably modified. The operating system is modified or created in accordance with the present disclosure as described.

Additionally, non-transitory computer readable media containing executable instructions that when executed cause a processor to perform operations including a method as provided herein are provided. In embodiments, the present disclosure includes an article of manufacture, such as a system or component thereof including a non-transitory computer-readable medium with instructions encoded thereon, the instructions configured to cause one or more processors to perform a method including: predicting a presence of a disease in accordance with the present disclosure. In embodiments the present disclosure includes non-transient computer readable medium with instructions encoded thereon, the instructions configured to cause one or more processors to perform a method including: predicting a presence of a disease in accordance with the present disclosure.

FIG. 2 illustrates a process flow 200 for predicting presence of a disease in a subject, according to another embodiment. At act 201, a plurality of parameters associated with the subject is received. The parameters associated with the subject may be obtained from a complete blood count/differential test performed for the subject. In particular, the parameters may include red blood cells (RBC) parameters, white blood cells (WBC) and platelet parameters. The RBC parameters include hemoglobin level, hematocrit, RBC size, hemoglobin level in individual RBC (normal, high low), mean corpuscular volume, mean corpuscular hemoglobin concentration, normal RBCs, number of RBCs with hemoglobin concentrations≥28 g/dL and ≤41 g/dL, number of RBCs with hemoglobin volumes≥60 fL and ≤120 fL hemoglobin distribution width, RBC mean corpuscular volume, RBC volume distribution width, total RBC count, hemoglobin content distribution width and mean of hemoglobin content. The RBC size-based parameters include RBC counts below 60 fL and above 120 fL. Additional parameters such as cell hemoglobin concentration mean, number of RBCs with hemoglobin concentrations greater than 41 g/dL, and number of RBCs with hemoglobin concentrations less than 28 g/dL are also considered. The parameters associated with the subject may be obtained from a complete blood count/differential test performed for the subject. In particular, the parameters may include red blood cells (RBC) parameters, white blood cells (WBC) and platelet parameters. In embodiments, the RBC parameters are preselected and include one or more of hemoglobin level, hematocrit, RBC size, hemoglobin level in individual RBC (normal, high low), mean corpuscular volume, mean corpuscular hemoglobin concentration, normal RBCs, number of RBCs with hemoglobin concentrations≥28 g/dL and ≤41 g/dL, number of RBCs with hemoglobin volumes≥60 fL and ≤120 fL hemoglobin distribution width, RBC mean corpuscular volume, RBC volume distribution width, total RBC count, hemoglobin content distribution width and mean of hemoglobin content, and combinations thereof.

The WBC parameters include WBC type such as lymphocytes, neutrophils, basophils, monocytes, eosinophils, polymorphonuclear cells, Large Unstained Cells (LUC), blasts and platelets. Additionally, WBC count, WBC percentage, ratio of neutrophils to lymphocytes, ratio of large unstained cells to lymphocytes, number of WBCs indicating pseudobasophilia, and WBC maturity parameters such as Delta Neutrophil Index are also considered. The parameters associated with the subject are indicative of inflammation and immune activation. Hence the parameters may be used to determine the presence of an infection in the subject. In embodiments, the WBC parameters are preselected. In embodiments, the WBC parameters include one or more of WBC type (such as lymphocytes, neutrophils, basophils, monocytes, eosinophils, polymorphonuclear cells, Large Unstained Cells (LUC), blasts and platelets, WBC count, WBC percentage, ratio of neutrophils to lymphocytes, ratio of large unstained cells to lymphocytes, number of WBCs indicating pseudobasophilia, WBC maturity parameters such as Delta Neutrophil Index, and combinations thereof.

In embodiments, platelet parameters are included and considered. Non-limiting platelet parameters include those shown in a plateletcrit, platelet volume, platelet count and platelet percentage.

At act 202, a threshold or threshold value for sensitivity and specificity associated with a trained machine learning model is determined for the desired use case. In embodiments, the threshold value is predetermined. In embodiments, the trained machine learning model is an ensemble of decision tree-based methods. For example, Extra Trees Classifier and Light Gradient Boosting Machine (LightGBM) may be used as the machine learning models. The ensemble of decision tree-based models is trained using hematology data associated with a plurality of subjects. The hematology data includes the WBC and RBC data obtained from Complete Blood Count/Differential test associated with the plurality of subjects. The machine learning models are trained with 5-fold cross validation and optimized for sensitivity. Extra Trees Classifier is an ensemble learning technique that improves accuracy and controls over-fitting by learning randomized decision trees on different data subsamples. Light Gradient Boosting Machine (LightGBM) is a boosting approach for decision tree learning in which the continuous feature values are discretized into bins for faster training and reduced memory usage. The different classifiers with the associated hyperparameters used in the ensemble are specified below (a-g). The weights for each classifier in the ensemble in the same order as listed [2, 1, 1, 2, 1, 1, 1].
a. ExtraTreesClassifier (bootstrap=False, class_weight=“balanced”, criterion=“gini”, max_features=“log 2”, min_samples_leaf=0.47421052631578947, min_samples_split=0.15052631578947367, n_estimators=600, oob_score-False)
b. LightGBM Classifier (boosting_type=“gbdt”, colsample_bytree-0.6933333333333332, learning_rate-0.05789894736842106, subsample_for_bin=190, max_depth=3, min_child_weight=9, min_data_in_leaf=0.03793724137931035, min_split_gain-0.15789473684210525, n_estimators=50, num_leaves=230, reg_alpha-0.2631578947368421, reg_lambda=0.7894736842105263, subsample=0.3963157894736842)
c. ExtraTreesClassifier (bootstrap=False, class_weight=“balanced”, criterion=“gini”, max_features=0.2, min_samples_leaf-0.01, min_samples_split=0.01, n_estimators=200, oob_score=False)
d. ExtraTreesClassifier (bootstrap=True, class_weight=“balanced”, criterion=“gini”, max_features-0.4, min_samples_leaf-0.01, min_samples_split=0.15052631578947367, n_estimators=10, oob_score=True)
e. ExtraTreesClassifier (bootstrap-False, class_weight=“balanced”, criterion=“gini”, max_features=“log 2”, min_samples_leaf-0.01, min_samples_split=0.056842105263157895, n_estimators=400, oob_score=False)
f. ExtraTreesClassifier (bootstrap=False, class_weight=“balanced”, criterion=“gini”, max_features=0.8, min_samples_leaf-0.01, min_samples_split=0.15052631578947367, n_estimators=25, oob_score=False)
g. ExtraTreesClassifier (Default parameters).
   (As used herein the term “Algorithm A” refers to the algorithm above).

In an embodiment, the threshold for sensitivity and/or specificity associated with the trained machine learning model is determined based on a requirement scenario associated with a healthcare provider. For example, in embodiments, the requirement scenario associated with the healthcare provider includes at least one of reduction of RT-PCR testing burden, reduction of infectious disease testing burden, substitution of RT-PCR testing, and/or determination of a need for testing of subjects. In the requirement scenario of reduction of RT-PCR testing burden, the algorithm may be used at a reasonably high sensitivity (e.g., 0.80-0.95), such that most positives will be predicted as positives and some negatives will be predicted as positives. By choosing the desired probability threshold, RT-PCR testing may be avoided in samples that are predicted negative by the algorithm. This approach may significantly reduce the RT-PCR testing burden in many labs while missing a small number of positives. This use case may be useful in the pandemic scenario. Similarly, for the requirement scenario of substitution of RT-PCR testing, the algorithm may be used at a very high sensitivity (e.g., 0.99-1.0), such that all positives will be predicted positive, and many negatives will be predicted as positives. In this high sensitivity scenario, RT-PCR testing may be avoided in samples that are predicted negative, without missing any true positives. This approach may reduce the RT-PCR testing burden in many labs/hospitals without missing any positives. This use case may be useful in the pandemic scenario. In case of the requirement scenario of determination of a need for testing of subjects, the algorithm may be used at a very high specificity (e.g., 0.99-1.0) such that all negatives will be predicted as negatives, but some positives will be flagged as positives. This approach may predict positives when none are suspected while positive flagging for negatives is minimized or absent. This use case may be useful in the endemic scenario in an emergency room.

At act 203, the presence of the disease in the subject is predicted using the trained machine learning model. Such prediction is made based on the sensitivity and specificity of the disease and the plurality of parameters associated with the subject. Binary classification models generate the probability of the input to be classified into categories (in the present case, COVID positive or negative). The final positive/negative callout is based on the threshold chosen. The default threshold in such models may be 0.5 (i.e., if the model outputs a probability of positive prediction as >0.5 for any input, the input is labeled positive, and if probability of positive prediction is <0.5, then it is labeled negative). Adjusting the threshold to a different value depending on the scenario enables curating the output from the model. For example, if a higher positive prediction rate (fewer false positives) is required, a higher threshold for sensitivity and specificity is chosen. However, if a higher negative prediction is required (fewer false negatives), a lower threshold for sensitivity and specificity is chosen.

At act 204, the prediction is outputted on an output unit 105.

For example, the prediction may be displayed on a display unit. In an alternate embodiment, the method includes determining a need for hospitalization of the subject based on the plurality of the parameters associated with the subject, using the trained machine learning model. The model is trained using the parameters associated with the plurality of subjects tested positive for the disease, wherein the parameters associated with the plurality of subjects is obtained within 2-3 days of the subjects testing positive for the disease. Additionally, the model is also trained with health data associated with the subjects that may be observed and collected over 7-10 days after the subjects testing positive for the disease.

In yet another embodiment, the method may include predicting an occurrence of long COVID-19 in the subject. The model is trained using the parameters associated with the plurality of subjects tested positive for the disease and health data associated with the subjects that may be observed and collected over the next months after the subjects testing positive for the disease.

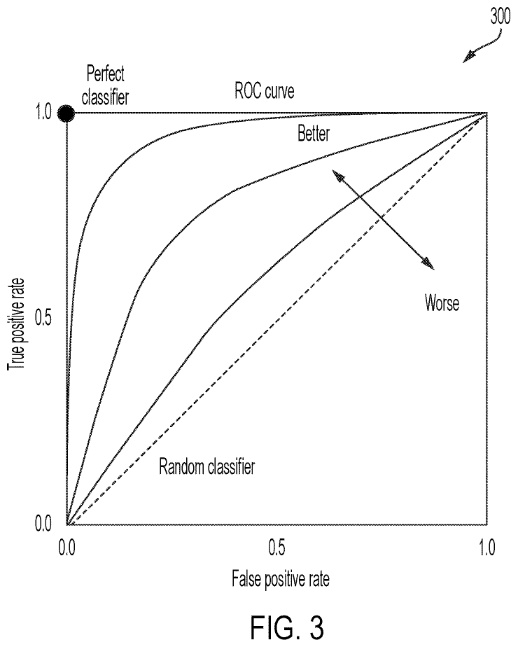

FIG. 3 illustrates a receiver operating characteristic (ROC) curve 300 for choosing threshold for sensitivity and specificity associated with a trained machine learning model for a requirement scenario, according to an embodiment. The ROC curve is a graphical plot that illustrates the diagnostic ability of a binary classification model as its discrimination threshold is varied. For various thresholds, ROC plots the true positive rate (sensitivity) against the false positive rate (probability of false alarm/1−specificity). The diagonal divides the ROC space. Points above the diagonal represent good classification results (better than random); points below the line represent bad results (worse than random).

### Treatment

In embodiments, the present disclosure includes a method of treating a subject in need thereof by determining that the subject is a subject in need thereof (such as has a pathogenic infection, or sepsis), and subsequently treating the subject. For examples, the methods of the present disclosure may be applied to diagnose a patient as positive for a pathogenic infection, sepsis, a viral infection such as COVID-19, followed by administering a therapeutic agent or compound to the subject in need thereof in a therapeutically effective amount. For example, a physician may administer a therapeutically effective amount of any drug, therapeutic agent or biologic suitable for treating the pathogenic infection, or disease state subsequent to diagnosis in accordance with the present disclosure. One non-limiting example of an agent suitable for treating a viral infection such as COVID-19 includes VEKLURY® brand remdesivir (100 MG for injection antiviral medication for treatment of patients hospitalized with COVID-19). See also, e.g., the international patent application WO 2017/049060 A1, and US Patent Application Publication No. 2021/0395345 entitled Methods for treating or preventing sars-cov-2 infections and covid-19 with anti-sars-cov-2 spike glycoprotein antibodies. The therapeutically effective amount of agent provided may be determined by a physician based on the subject's response, comorbidities, and the like. In embodiments, a therapeutic compound is administered in an effective amount to a subject in a diseased state. In embodiments, a therapeutically effective amount of a compound is administered to a subject in need thereof in an amount sufficient to change the natural or presenting state of the subject.

In embodiments, a subject may be in a diseased state due to infection with an RNA virus. Non-limiting examples of an RNA virus include segmented, single-stranded, negative sense RNA viruses (e.g., Orthomyxoviruses); non-segmented, single-stranded, negative sense RNA viruses (Mononegavirales); non-segmented, single-stranded, positive sense RNA viruses (e.g., Coronaviruses); ambisense RNA viruses (e.g., Bunyavirus and Arenavirus, e.g., Lassa virus, Junin virus, Machupo virus, and lymphocytic choriomeninfitis virus); and double-stranded RNA viruses (e.g., Reoviruses). Other viruses causing disease include a segmented, single-stranded, negative sense RNA viruses; a non-segmented, single-stranded, negative sense RNA virus; a non-segmented, single-stranded, positive sense RNA viruses; or a double-stranded RNA viruses (e.g., Reoviruses). Additional RNA viruses include: Picornaviruses, togaviruses (e.g, Sindbis virus), flaviviruses, and coronaviruses. The virus may be any type, species, and/or strain of picornavirus, togavirus flavivirus, and coronavirus. Further disease-causing viruses include: respiratory syncytial virus (RSV), influenza virus (influenza A virus, influenza B virus, or influenza C virus), human metapneumovirus (HMPV), rhinovirus, parainfluenza virus, SARS Coronavirus, human immunodeficiency virus (HIV), hepatitis virus (A, B, C), ebola virus, herpes virus, rubella, variola major, and variola minor.

In certain embodiments, a therapeutic composition is administered to a subject who has been diagnosed with a disease caused by infection with a virus, e.g., the patient has been infected by respiratory syncytial virus (RSV), influenza virus (influenza A virus, influenza B virus, or influenza C virus), human metapneumovirus (HMPV), rhinovirus, parainfluenza virus, SARS Coronavirus, human immunodeficiency virus (HIV), hepatitis virus (A, B, C), ebola virus, herpes virus, rubella, variola major, and/or variola minor.

In certain embodiments, the disease treated in accordance with the methods described herein is a disease caused by bacterial infection. Non-limiting examples of disease-causing bacteria include: Streptococcus pneumoniae, Mycobacterium tuberculosis, Chlamydia pneumoniae, Bordetella pertussis, Mycoplasma pneumoniae, Haemophilus influenzae, Moraxella catarrhalis, Legionella, Pneumocystis proveci, Chlamydia psittaci, Chlamydia trachomans, Bacillus anthracis, and Francisella tularensis, Borrelia burgdorferi, Salmonella, Yersinia pestis, Shigella, E. coli, Corynebacterium diphtheriae, and Treponema pallidum.

In certain embodiments, a composition is administered to a patient who has been diagnosed with a disease caused by infection with a bacteria, e.g., the patient has been infected by Streptococcus pneumoniae, Mycobacterium tuberculosis, Chlamydia pneumoniae, Bordetella pertussis, Mycoplasma pneumoniae, Haemophilus influenzae, Moraxella catarrhalis, Legionella, Pneumocystis jiroveci, Chlamydia psittaci, Chlamydia trachomatis, Bacillus anthracis, and Francisella tularensis, Borrelia burgdorferi, Salmonella, Yersinia pestis, Shigella, E. coli, Corynebacterium diphtheriae, and/or Treponema pallidum.

In certain embodiments, a composition is administered to a patient diagnosed with a disease caused by a fungal infection, e.g., the patient has been infected by Blastomyces, Paracoccidioides, Sporothrix, Cryptococcus, Candida, Aspergillus, Histoplasma, Cryptococcus, Bipolaris, Cladophialophora, Cladosporium, Drechslera, Exophiala, Fonsecaea, Phialophora, Xylohypha, Ochroconis, Rhinocladiella, Scolecobasidium, and/or Wangiella.

In certain embodiments, a composition is administered to a patient who has been diagnosed with a disease caused by infection with a yeast, e.g., the patient has been infected by Aciculoconidium, Botryouscus, Brettanomyces, Bullera, Bulleromyces, Candida, Citeromyces, Clavispora, Cryptococcus, Cystofilobasidium, Debaromyces, Debaryomyces, Dekkera, Dipodaseus, Endomyces, Endomycopsis, Erythrobasidium, Fellomyces, Filobasidium, Guilliermondella, Hanseniaspora, Hansenula, Hasegawaea, Hyphopichia, Issatchenkia, Kloeckera, Kluyveromyces, Komagataella, Leucosporidium, Lipomyces, Lodderomyces, Malassezia-Mastigomyces, Metschnikowia, Mrakia, Nadsonia, Octosporomyces, Oosporidium, Pachysolen, Petasospora, Phaffia, Pichia, Pseudozyma, Rhodosporidium, Rhodotorula, Saccharomyces, Saccharomycodes, Saccharomycopsis, Schizoblastosporion, Schizosaccharomyces, Schwanniomyces, Selenotila, Sirobasidium, Sporidiobolus, Sporobolomyces, Stephanoascus, Sterigmatomyces, Syringospora, Torulaspora, Torulopsis, Tremelloid, Trichosporon, Trigonopsis, Udeniomyces, Waltomyces, Wickerhamia, Williopsis, Wingea, Yarrowia, Zygofabospora, Zygolipomyces, and/or Zygosaccharomyces.

In certain embodiments, a composition is administered to a subject infected with a parasite, e.g., the patient has been infected by Babesia, Cryptosporidium, Entamoeba histolytica, Leishmania, Giardia lamblia, Plasmodium, Toxoplasma, Trichomonas, Trypanosoma, Ascaris, Cestoda, Ancylostoma, Brugia, Fasciola, Trichinella, Schistosoma, Taenia, Cimicidae, Pediculus, and/or Sarcoptes. See additional information in U.S. patent publication no. 20170321192 (herein entirely incorporated by reference).

### EXEMPLARY NUMBERED EMBODIMENTS

Embodiment 1: A system (100) for predicting presence of a disease in a subject, the system (100) including: one or more processors (101); a medical database (112) coupled to the one or more processors (101), the medical database (112) including patient data; and a disease prediction module (110) configured to: receive a plurality of parameters associated with the subject; determine a threshold for sensitivity and/or specificity associated with a trained machine learning model; predict using the trained machine learning model the presence of the disease in the subject based on a specific threshold of the model and the plurality of parameters associated with the subject; and output the prediction on an output unit (105).

Embodiment 2: A system (100) for predicting presence of a disease in a subject, the system (100) including: one or more processors (101); a medical database (112) coupled to the one or more processors (101), the medical database (112) including patient data; and a disease prediction module (110) configured to: receive a plurality of parameters associated with the subject; determine a threshold for sensitivity and/or specificity associated with a trained machine learning model; predict using the trained machine learning model the presence of the disease in the subject based on a specific threshold of the model and the plurality of parameters associated with the subject; and output the prediction on an output unit (105), wherein the threshold or threshold value for sensitivity and/or specificity associated with the trained machine learning model is determined based on a requirement scenario associated with a healthcare provider.

Embodiment 3: A system (100) for predicting presence of a disease in a subject, the system (100) including: one or more processors (101); a medical database (112) coupled to the one or more processors (101), the medical database (112) including patient data; and a disease prediction module (110) configured to: receive a plurality of parameters associated with the subject; determine a threshold for sensitivity and/or specificity associated with a trained machine learning model; predict using the trained machine learning model the presence of the disease in the subject based on a specific threshold or threshold value of the model and the plurality of parameters associated with the subject; and output the prediction on an output unit (105), wherein the threshold or threshold value for sensitivity and/or specificity associated with the trained machine learning model is determined based on a requirement scenario associated with a healthcare provider, and wherein the requirement scenario associated with the healthcare provider includes at least one of reduction of RT-PCR testing burden, substitution of RT-PCR testing, and/or determination of a need for testing of subjects.

Embodiment 4: A system (100) for predicting presence of a disease in a subject, the system (100) including: one or more processors (101); a medical database (112) coupled to the one or more processors (101), the medical database (112) including patient data; and a disease prediction module (110) configured to: receive a plurality of parameters associated with the subject; determine a threshold for sensitivity and/or specificity associated with a trained machine learning model; predict using the trained machine learning model the presence of the disease in the subject based on a specific threshold of the model and the plurality of parameters associated with the subject; and output the prediction on an output unit (105), wherein the plurality of parameters associated with the subject includes red blood cells (RBC) parameters and white blood cells (WBC) parameters.

Embodiment 5: A system (100) for predicting presence of a disease in a subject, the system (100) including: one or more processors (101); a medical database (112) coupled to the one or more processors (101), the medical database (112) including patient data; and a disease prediction module (110) configured to: receive a plurality of parameters associated with the subject; determine a threshold for sensitivity and/or specificity associated with a trained machine learning model; predict using the trained machine learning model the presence of the disease in the subject based on a specific threshold of the model and the plurality of parameters associated with the subject; and output the prediction on an output unit (105), wherein the plurality of parameters associated with the subject includes red blood cells (RBC) parameters and white blood cells (WBC) parameters, and wherein the RBC parameters include one or more of hemoglobin level, hematocrit, RBC size, hemoglobin level in individual RBC, mean corpuscular volume, mean corpuscular hemoglobin concentration, normal RBCs, number of RBCs with hemoglobin concentrations≥28 g/dL and ≤41 g/dL, number of RBCs with hemoglobin volumes≥60 fL and ≤120 fL, hemoglobin distribution width, RBC mean corpuscular volume, RBC volume distribution width, total RBC count, hemoglobin content distribution width and mean of hemoglobin content.

Embodiment 6: A system (100) for predicting presence of a disease in a subject, the system (100) including: one or more processors (101); a medical database (112) coupled to the one or more processors (101), the medical database (112) including patient data; and a disease prediction module (110) configured to: receive a plurality of parameters associated with the subject; determine a threshold for sensitivity and/or specificity associated with a trained machine learning model; predict using the trained machine learning model the presence of the disease in the subject based on a specific threshold of the model and the plurality of parameters associated with the subject; and output the prediction on an output unit (105), wherein the plurality of parameters associated with the subject includes red blood cells (RBC) parameters and white blood cells (WBC) parameters, wherein the WBC parameters include one or more of WBC type, WBC count, WBC percentage, ratio of neutrophils to lymphocytes, ratio of large unstained cells to lymphocytes, number of WBCs indicating pseudobasophilia, and WBC maturity parameters.

Embodiment 7: A system (100) for predicting presence of a disease in a subject, the system (100) including: one or more processors (101); a medical database (112) coupled to the one or more processors (101), the medical database (112) including patient data; and a disease prediction module (110) configured to: receive a plurality of parameters associated with the subject; determine a threshold for sensitivity and/or specificity associated with a trained machine learning model; predict using the trained machine learning model the presence of the disease in the subject based on a specific threshold of the model and the plurality of parameters associated with the subject; and output the prediction on an output unit (105), wherein the plurality of parameters associated with the subject includes red blood cells (RBC) parameters and white blood cells (WBC) parameters, and wherein the RBC parameters include one or more of hemoglobin level, hematocrit, RBC size, hemoglobin level in individual RBC, mean corpuscular volume, mean corpuscular hemoglobin concentration, normal RBCs, number of RBCs with hemoglobin concentrations≥28 g/dL and ≤41 g/dL, number of RBCs with hemoglobin volumes≥60 fL and ≤120 fL, hemoglobin distribution width, RBC mean corpuscular volume, RBC volume distribution width, total RBC count, hemoglobin content distribution width and mean of hemoglobin content, wherein the system, further including cell hemoglobin concentration mean, number of RBCs with hemoglobin concentrations greater than 41 g/dL, and number of RBCs with hemoglobin concentrations less than 28 g/dL.

Embodiment 8: A system (100) for predicting presence of a disease in a subject, the system (100) including: one or more processors (101); a medical database (112) coupled to the one or more processors (101), the medical database (112) including patient data; and a disease prediction module (110) configured to: receive a plurality of parameters associated with the subject; determine a threshold for sensitivity and/or specificity associated with a trained machine learning model; predict using the trained machine learning model the presence of the disease in the subject based on a specific threshold of the model and the plurality of parameters associated with the subject; and output the prediction on an output unit (105), wherein the trained machine learning model is an ensemble of decision tree-based methods.

Embodiment 9: A system (100) for predicting presence of a disease in a subject, the system (100) including: one or more processors (101); a medical database (112) coupled to the one or more processors (101), the medical database (112) including patient data; and a disease prediction module (110) configured to: receive a plurality of parameters associated with the subject; determine a threshold for sensitivity and/or specificity associated with a trained machine learning model; predict using the trained machine learning model the presence of the disease in the subject based on a specific threshold of the model and the plurality of parameters associated with the subject; and output the prediction on an output unit (105), wherein the disease prediction module (110) is further configured to predict a need for hospitalization of the subject based on the plurality of the parameters associated with the subject, using the trained machine learning model.

Embodiment 10: A system (100) for predicting presence of a disease in a subject, the system (100) including: one or more processors (101); a medical database (112) coupled to the one or more processors (101), the medical database (112) including patient data; and a disease prediction module (110) configured to: receive a plurality of parameters associated with the subject; determine a threshold for sensitivity and/or specificity associated with a trained machine learning model; predict using the trained machine learning model the presence of the disease in the subject based on a specific threshold of the model and the plurality of parameters associated with the subject; and output the prediction on an output unit (105), wherein the disease prediction module (110) is further configured to predict occurrence of long COVID-19 in the subject.

Embodiment 11: A method (200) of predicting presence of a disease in a subject, the method (200) including: receiving a plurality of parameters associated with the subject; determining a threshold for sensitivity and/or specificity associated with a trained machine learning model; predicting using the trained machine learning model the presence of the disease in the subject based on a specific threshold of the model and the plurality of parameters associated with the subject; and outputting the prediction on an output unit.

Embodiment 12: A method (200) of predicting presence of a disease in a subject, the method (200) including: receiving a plurality of parameters associated with the subject; determining a threshold for sensitivity and/or specificity associated with a trained machine learning model; predicting using the trained machine learning model the presence of the disease in the subject based on a specific threshold of the model and the plurality of parameters associated with the subject; and outputting the prediction on an output unit, wherein the threshold for sensitivity and/or specificity associated with the trained machine learning model is determined based on a requirement scenario associated with a healthcare provider.

Embodiment 13: A method (200) of predicting presence of a disease in a subject, the method (200) including: receiving a plurality of parameters associated with the subject; determining a threshold for sensitivity and/or specificity associated with a trained machine learning model; predicting using the trained machine learning model the presence of the disease in the subject based on a specific threshold of the model and the plurality of parameters associated with the subject; and outputting the prediction on an output unit, wherein the threshold for sensitivity and/or specificity associated with the trained machine learning model is determined based on a requirement scenario associated with a healthcare provider, and wherein the requirement scenario associated with the healthcare provider includes at least one of reduction of RT-PCR testing burden, substitution of RT-PCR testing, and/or determination of a need for testing of subjects.

Embodiment 14: A method (200) of predicting presence of a disease in a subject, the method (200) including: receiving a plurality of parameters associated with the subject; determining a threshold for sensitivity and/or specificity associated with a trained machine learning model; predicting using the trained machine learning model the presence of the disease in the subject based on a specific threshold of the model and the plurality of parameters associated with the subject; and outputting the prediction on an output unit, wherein the disease is COVID-19.

Embodiment 15: A method (200) of predicting presence of a disease in a subject, the method (200) including: receiving a plurality of parameters associated with the subject; determining a threshold for sensitivity and/or specificity associated with a trained machine learning model; predicting using the trained machine learning model the presence of the disease in the subject based on a specific threshold of the model and the plurality of parameters associated with the subject; and outputting the prediction on an output unit, further including: predicting a need for hospitalization of the subject based on the plurality of the parameters associated with the subject, using the trained machine learning model; and predicting an occurrence of long COVID-19 in the subject.

Embodiment 16: A method (200) of predicting presence of a disease in a subject, the method (200) including: receiving a plurality of parameters associated with the subject; determining a threshold for sensitivity and/or specificity associated with a trained machine learning model; predicting using the trained machine learning model the presence of the disease in the subject based on a specific threshold of the model and the plurality of parameters associated with the subject; and outputting the prediction on an output unit, wherein the disease is COVID-19.

Embodiment 17: A method (200) of predicting presence of a disease in a subject, the method (200) including: receiving a plurality of parameters associated with the subject; determining a threshold for sensitivity and/or specificity associated with a trained machine learning model; predicting using the trained machine learning model the presence of the disease in the subject based on a specific threshold of the model and the plurality of parameters associated with the subject; and outputting the prediction on an output unit, further including: predicting a need for hospitalization of the subject based on the plurality of the parameters associated with the subject, using the trained machine learning model; and predicting an occurrence of viral, bacterial, or fungal infection in the subject.

Embodiment 18: An article of manufacture, such as a system or component thereof including a non-transitory computer-readable medium with instructions encoded thereon, the instructions configured to cause one or more processors to perform a method of Embodiments 15-17 above.

Embodiment 19: In embodiments the present disclosure includes non-transient computer readable medium with instructions encoded thereon, the instructions configured to cause one or more processors to perform a method including: predicting presence of a disease in a subject, the method (200) including: receiving a plurality of parameters associated with the subject; determining a threshold for sensitivity and/or specificity associated with a trained machine learning model; predicting using the trained machine learning model the presence of the disease in the subject based on a specific threshold of the model and the plurality of parameters associated with the subject; and outputting the prediction on an output unit, further including: predicting a need for hospitalization of the subject based on the plurality of the parameters associated with the subject, using the trained machine learning model; and predicting an occurrence of viral, bacterial, or fungal infection in the subject.

Embodiment 20: In embodiments the present disclosure includes non-transient computer readable medium with instructions encoded thereon, the instructions configured to cause one or more processors to perform a method including predicting presence of a disease in a subject, the method (200) including: receiving a plurality of parameters associated with the subject; determining a threshold for sensitivity and/or specificity associated with a trained machine learning model; predicting using the trained machine learning model the presence of the disease in the subject based on a specific threshold of the model and the plurality of parameters associated with the subject; and outputting the prediction on an output unit, further including: predicting a need for hospitalization of the subject based on the plurality of the parameters associated with the subject, using the trained machine learning model; and predicting an occurrence of sepsis in the subject.

Embodiment 21: An article of manufacture including a non-transitory computer-readable medium with instructions encoded thereon, the instructions configured to cause one or more processors to perform a method including: predicting a presence of a disease in accordance with an embodiment of the present disclosure.

Embodiment 22: A non-transient computer readable medium with instructions encoded thereon, the instructions configured to cause one or more processors to perform a method including: predicting a presence of a disease in accordance with at least one embodiment of the present disclosure.

Embodiment 23: A method of treating a subject in need thereof including predicting a presence of a disease in a subject in accordance with an embodiment of the present disclosure and subsequently treating the subject in need thereof. In embodiments, treating includes administering to the subject a therapeutically effective amount of a composition.

Embodiment 24: A method of treating a subject in need thereof including predicting presence of a disease in a subject, the method (200) including: receiving a plurality of parameters associated with the subject; determining a threshold for sensitivity and/or specificity associated with a trained machine learning model; predicting using the trained machine learning model the presence of the disease in the subject based on a specific threshold of the model and the plurality of parameters associated with the subject; and outputting the prediction on an output unit, and subsequently treating the subject in need thereof. In embodiments, treating includes administering to the subject a therapeutically effective amount of a composition. In embodiments, the disease is characterized as viral disease, bacterial disease, COVID-19, long-Covid, or the like.

Embodiment 25: In embodiments, the present disclosure includes a system for predicting a presence of a disease in a population. In one aspect of the present disclosure, the system includes a processor and a memory. Additionally, the memory includes a disease prediction module configured to receive a plurality of parameters associated with one or more subjects and determine a threshold for sensitivity and/or specificity associated with a trained machine learning model. Additionally, the module is configured to predict using the trained machine learning model the presence of the disease in one or more subjects or a population of subjects and the plurality of parameters associated with the subject or population of subjects and output the prediction on an output unit.

### EXAMPLES

### Example 1

The present example describes a COVID-19 detection algorithm based on CBC/Diff hematology data. In embodiments, present disclosure includes a multiparametric machine learning algorithm to run on, among other things, a hematology instrument and provide a COVID-19 flag along with the CBC/Diff data. In embodiments, the algorithm may be run at different sensitivity and specificity values based on the intended use case, in a pandemic or an endemic situation. The training data used in the algorithm is based on an unvaccinated cohort from 2020. Additional data from more varied positive and negative individuals may be used to improve the robustness of the algorithm.

Hematology tests such as CBC/Diff (Complete Blood Count with Differential) are ubiquitously performed for overall health monitoring. In embodiments, the present disclosure combines CBC/Diff results from the Siemens Healthineers ADVIA® 2120i Hematology System with a multiparametric machine learning algorithm for rapid flagging of COVID-19 (Coronavirus Disease 2019) positivity. Diagnostic data generation and running the algorithm on the data all happen on the same instrument, providing easy data access. Additionally, the algorithm may also run on a connected lab information system. Such an algorithm may be valuable when access to COVID testing is limited and for predicting COVID-19 in unsuspected individuals thus prompting a confirmatory test. Here, various pandemic and endemic applications for this algorithm are demonstrated.

Methods. CBC/Diff data for 309 COVID-19 positives and 245 negatives were obtained from a 2020 pre-vaccinated cohort from Mumbai, India. Using this data, machine learning classification models were trained with 5-fold cross-validation for distinguishing COVID-19 positives from negatives.

Results. The final ML model was an ensemble of decision tree-based methods, with an accuracy of 74% and AUROC of 0.79 on the test set. The most significant features include eosinophil and lymphocyte counts that have been reported earlier. The model was able to predict hospitalized COVID-19 patients more accurately (92% correctly classified) than non-hospitalized patients (70% correctly classified).

Conclusion. A predictive algorithm, method, and system is provided for rapid COVID-19 flagging using data from routine CBC testing. This algorithm is useful in pandemic or endemic scenarios as an alternate to testing, reducing testing burden and flagging unsuspected positives.

Discussion Hematology data provides a snapshot of the state of health of a patient and represents the host's response to an infection. Additionally, a hematology test such as a CBC/Diff is commonly prescribed before any disease suspicion and is thus an ideal platform for flagging and stratifying a possible infection or a health condition, thus enabling an earlier intervention and streamlining different medical resources (See e.g., Barnes P W, McFadden S L, Machin S J, Simson E. The international consensus group for hematology review: suggested criteria for action following automated CBC and WBC differential analysis. Laboratory hematology: official publication of the International Society for Laboratory Hematology 2005; 11 2:83-90). Here a CBC/Diff test performed on the Siemens Healthineers ADVIA® 2120i Hematology System was combined with a multiparametric AI algorithm to flag COVID-19 positivity in individuals.

Studies have reported hematologic characteristics like eosinopenia, and lymphopenia associated with COVID-19 positivity and prognosis (See e.g., Frater J L, Zini G, d'Onofrio G, Rogers H J. COVID-19 and the clinical hematology laboratory. International Journal of Laboratory Hematology 2020; 42:11-8; Guan W-j, Ni Z-y, Hu Y, Liang W-h, Ou C-q, He J-x, Liu L, et al. Clinical characteristics of coronavirus disease 2019 in China. New England journal of medicine 2020; 382:1708-20; Henry B M, De Oliveira M H S, Benoit S, Plebani M, Lippi G. Hematologic, biochemical and immune biomarker abnormalities associated with severe illness and mortality in coronavirus disease 2019 (COVID-19): a meta-analysis}. Clinical Chemistry and Laboratory Medicine (CCLM) 2020; 58:1021; Jiang S-Q, Huang Q-F, Xie W-M, Lv C, Quan X-Q. The association between severe COVID-19 and low platelet count: evidence from 31 observational studies involving 7613 participants. British journal of haematology 2020: e29-e33; and Kermali M, Khalsa R K, Pillai K, Ismail Z, Harky A. The role of biomarkers in diagnosis of COVID-19—A systematic review. Life sciences 2020; 254:117788). Some hematology parameters also show mixed trends in different studies, possibly due to cohort dependence of parameter values. However, it is difficult for physicians to identify COVID-19 positivity by evaluating a single parameter in isolation or even a combination of parameters. Multiparametric machine learning (ML) algorithms enable modeling of complex relationships between multiple predictive parameters and generate predictions more accurately and efficiently. Unlike multi-parametric studies that combine parameters measured on multiple instruments (See e.g., Kukar M, Gunčar G, Vovko T, Podnar S, Černelč P, Brvar M, Zalaznik M, et al. COVID-19 diagnosis by routine blood tests using machine learning. Scientific reports 2021; 11:1-9), the present disclosure uses multiple hematology parameters measured/derived using a single instrument to make predictions. This precludes the need to aggregate data from multiple sources and allows easy data access without any missing values, a common problem in such multiparametric algorithms.

COVID-19 testing using RT-PCR (reverse transcriptase-polymerase chain reaction) and antigen tests have scaled up massively since the onset of the pandemic, however, there are multiple use cases where a CBC/Diff based predictive algorithm may be applied in the clinical value chain both in the pandemic and endemic scenarios. First, it may act as a substitute for testing in countries dealing with unavailability or shortages of such tests. Second, quick flagging by a highly sensitive hematology-based machine learning (ML) algorithm may reduce the testing burden in a pandemic scenario. Third, an algorithm with high negative predictive value (NPV) may reduce the number of patients undergoing testing before procedures like surgery, thereby reducing time to patient care. Fourth, in an endemic scenario, an algorithm with high specificity may be used for flagging asymptomatic individuals who may then be tested to confirm the infection. The same ML algorithm with different classification thresholds may be used in multiple scenarios described as above.

A hematology based multiparametric ML algorithm may be used for COVID flagging within seconds to minutes. The reduced time to result may avoid unnecessary quarantining of negatives and rapid access to care for patients that need to undergo procedures like surgery in a pandemic scenario and also be used for flagging unsuspected positive during an endemic scenario.

The present disclosure includes a multi-parametric machine learning algorithm to predict COVID-19 positivity in individuals using CBC/Diff data derived from a single instrument. In embodiments, the multiparametric ML algorithm predicts COVID-19 positivity in individuals with an accuracy of 74% and AUROC (area under the receiver operating characteristic curve) of 0.79. Furthermore, in embodiments, the algorithm predicts positive hospitalized patients with 91.7% accuracy, giving us confidence in our algorithm performance and the potential of the parameters selected.

### Materials and Methods

### Datasets

CBC/Diff data was obtained from two ADVIA® 2120i instruments installed in Dr. Jariwala Laboratory & Diagnostics in Mumbai (India) for this analysis. The present disclosure includes a retrospective study on a cohort of 309 positive and 245 COVID-19 negative individuals, determined using RT-PCR tests. Only anonymized data was sent to the researchers, which included information on the sex and age of the individuals and the disease outcome in COVID-19 positive individuals (whether they were hospitalized/admitted to the ICU/survived or died). The COVID-19 positives and negatives were concurrently measured during the first wave of infection in India (May 2020-January 2021) before any vaccination drives. The COVID-19 negatives included in this study were individuals receiving medical attention for other conditions during the first wave of the pandemic. Of the 309 positives, 60 were hospitalized at the time of CBC/Diff testing, out of which 29 were in the ICU and 53 survived the infection. The demographics and blood cell counts of COVID-19 positive and negative individuals included in this study are summarized in Table 1.

TABLE 1
Demographics and blood cell counts of COVID-19-positive
and negative individuals used in this study.

| | Positives | Negatives |
|---|---|---|
| | (201 males, 108 | (116 males, 129 |
| | females) | females) |
| | Median (IQR) | Median (IQR) |
| Age | 53 (41-64) | 44 (31-59) |
| WBC (×10³ cells/μL) | 6.18 (4.89-7.84) | 8.15 (6.41-10.11) |
| Neutrophils (×10³ cells/μL) | 3.98 (2.82-5.09) | 4.97 (3.81-6.84) |
| Lymphocytes (×10³ | 1.44 (1.01-1.91) | 1.94 (1.4-2.45) |
| cells/μL) | | |
| Monocytes (×10³ cells/μL) | 0.37 (0.27-0.5) | 0.4 (0.3-0.5) |
| Eosinophils (×10³ cells/μL) | 0.05 (0.02-0.14) | 0.16 (0.07-0.32) |
| Large unstained cells (×10³ | 0.15 (0.11-0.21) | 0.21 (0.15-0.27) |
| cells/μL) | | |
| Basophils (×10³ cells/μL) | 0.06 (0.04-0.09) | 0.07 (0.05-0.1) |
| Platelets (×10³ cells/μL) | 239 (192-295) | 280 (222-330) |
| Red cell count (×10⁶ | 4.3 (3.97-4.65) | 4.2 (3.9-4.5) |
| cells/μL) | | |
| HGB (g/dL) | 13.5 (12.3-14.7) | 12.7 (11.6-14.1) |
| RDW | 13.7 (13.2-14.5) | 14 (13.3-15.1) |

### Parameter Selection

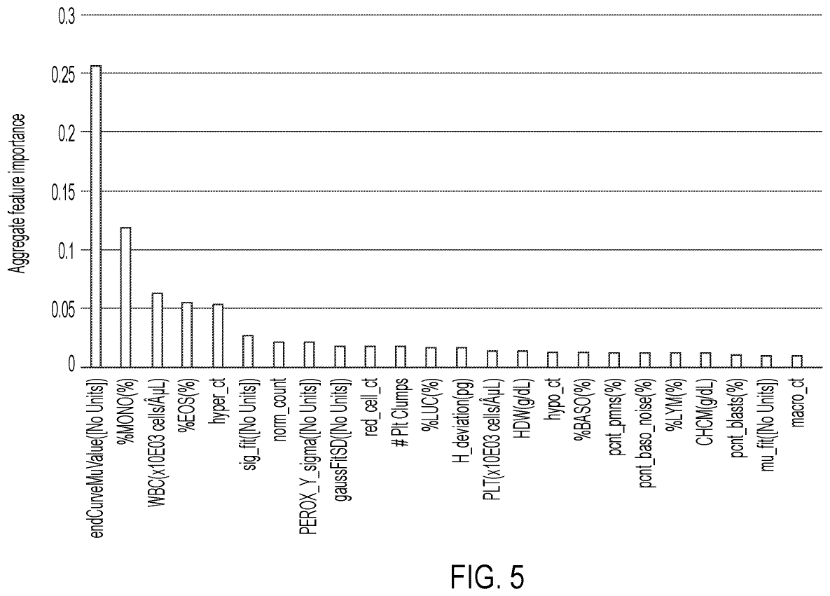

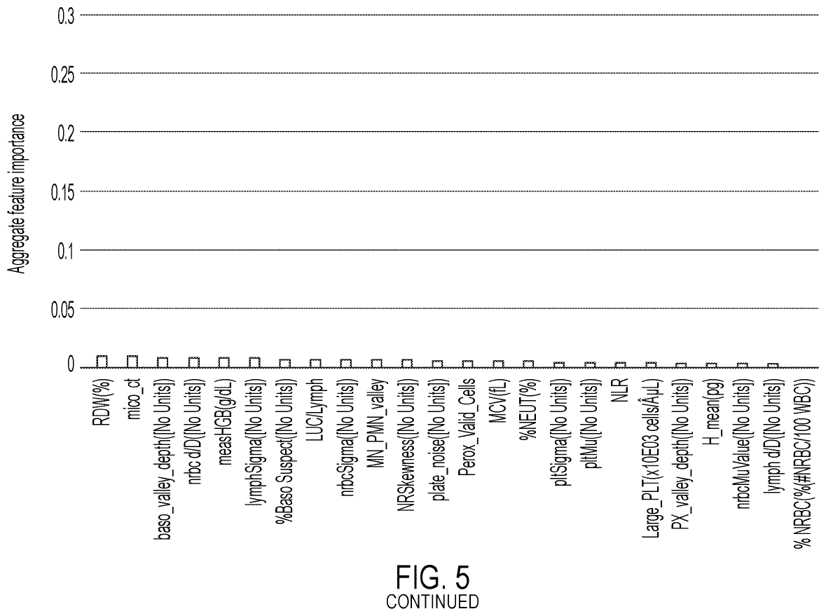

The starting dataset included all the parameters included in the ADVIA 2120i CBC/Diff raw data set. Parameters were eliminated using various criteria, e.g., where the median value did not vary significantly between the positives and the negatives (done using box plots and p-values for Kruskal-Wallis H-test), parameters that measured only instrument characteristics, etc. Redundant and highly correlated parameters were removed (Spearman correlation coefficient>0.9). Finally, a set of 48 parameters were identified (See FIG. 5) that included both raw and calculated patient parameters. All 48 parameters have numerical values, and there are no missing values. No patient demographic information or information identifying the source instrument (instrument 1 or 2) were included while training the algorithm.

### Algorithm

After parameter identification, Microsoft Azure AutoML (https://www.microsoft.com/en-us/research/project/automl/) was used to train classification models to distinguish positives and negatives. 80% of the data (247 positives, 196 negatives) was used for training the models using with 5-fold cross validation and used the remaining 20% data (62 positives, 49 negatives) as the test set. Azure AutoML uses the dataset to automatically train many different models, and model training may be optimized based on different metrics. When optimized for sensitivity, an excellent model for the dataset is a voting ensemble of multiple tree-based classifiers. The different classifiers with the associated hyperparameters used in the ensemble are specified below (a-g). The weights for each classifier in the ensemble in the same order as listed are [2, 1, 1, 2, 1, 1, 1].
h. ExtraTreesClassifier (bootstrap=False, class_weight=“balanced”, criterion=“gini”, max_features=“log 2”, min_samples_leaf=0.47421052631578947, min_samples_split=0.15052631578947367, n_estimators=600, oob_score=False)
i. LightGBM Classifier (boosting_type-“gbdt”, colsample_bytree-0.6933333333333332, learning_rate-0.05789894736842106, subsample_for_bin-190, max_depth-3, min_child_weight=9, min_data_in_leaf=0.03793724137931035, min_split_gain-0.15789473684210525, n_estimators=50, num_leaves=230, reg_alpha-0.2631578947368421, reg_lambda-0.7894736842105263, subsample=0.3963157894736842)
j. ExtraTreesClassifier (bootstrap=False, class_weight-“balanced”, criterion=“gini”, max_features-0.2, min_samples_leaf-0.01, min_samples_split=0.01, n_estimators=200, oob_score=False)
k. ExtraTreesClassifier (bootstrap=True, class_weight=“balanced”, criterion=“gini”, max_features-0.4, min_samples_leaf-0.01, min_samples_split=0.15052631578947367, n_estimators=10, oob_score=True)
l. ExtraTreesClassifier (bootstrap=False, class_weight=“balanced”, criterion=“gini”, max_features=“log 2”, min_samples_leaf-0.01, min_samples_split=0.056842105263157895, n_estimators=400, oob_score=False)
m. ExtraTreesClassifier (bootstrap=False, class_weight=“balanced”, criterion=“gini”, max_features=0.8, min_samples_leaf-0.01, min_samples_split=0.15052631578947367, n_estimators=25, oob_score=False)
n. ExtraTreesClassifier (Default parameters).

The trained ensemble model was downloaded from Microsoft Azure. This gives excellent flexibility in using the model, e.g., in evaluation of the model performance on the test set and in deployment.

### Results

The performance of the model was evaluated based on different metrics including prediction accuracy, AUROC, sensitivity and specificity. Next, the feature importance obtained using MIMIC LightGBM explainer on the training data was presented. The MIMIC LightGBM explainer approximates the complex, black-box ensemble model obtained from Azure AutoML using a LightGBM surrogate model to obtain feature/parameter importance values. A comparison of the results from our 48-parameter model with other models learned using the most important parameters was performed.

### Different Use Cases for the Algorithm.

### Model Evaluation

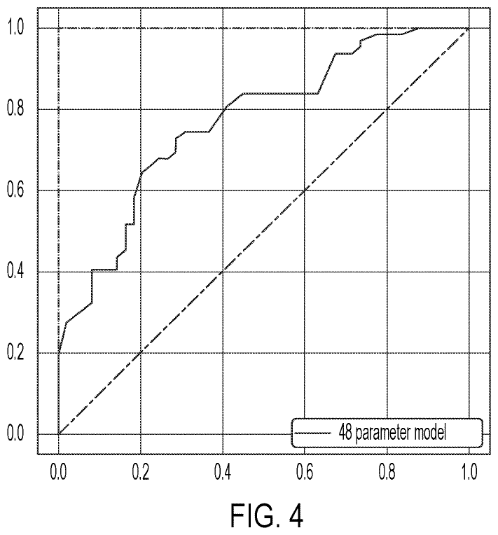

The entire dataset was divided into separate training (80% of total data) and test sets (remaining 20% data). The model was trained using 5-fold cross-validation on the training set and the model performance was evaluated on the test set. The test data set consisted of 62 positives and 49 negatives. The model performed with an accuracy of 74%, sensitivity of 0.73, specificity of 0.74 and AUROC of 0.79 on the test data (all metrics are reported using a default classification threshold of 0.5). FIG. 4 shows the ROC (Receiver operating curve) curve computed using the test data. Since the positives and negatives in the test are balanced, AUROC is a good performance metric for validation.

Out of 62 positives in the test set, 12 were hospitalized whereas the remaining 50 were not. It was observed that the hospitalized positives were more accurately predicted than non-hospitalized positives (Table 2).

TABLE 2
Prediction accuracy for hospitalized positives, non-hospitalized
positives, and COVID-19 negatives in the test set

| | Positive- | Positive-not | | |
|---|---|---|---|---|
| | hospitalized | hospitalized | Negative | Total |
| Accuracy | 92% | 70% | 74% | 74% |
| (Correctly | (11/12) | (35/50) | (36/49) | (82/111) |
| predicted/Total) | | | | |

This gives increased confidence in the selected parameters. The effect of age and sex was also observed on algorithm performance (See Supplementary Table 1 and Supplementary Table 2 below) and found that the algorithm prediction showed no bias for age or sex.

### Feature Importance

Here, the model interpretations obtained using the MIMIC LightGBM explainer on the training data are presented. FIG. 5 shows the features in decreasing order of importance values, as computed by the model explainer.

Comparison with Models Generated with Top Parameters

The importance of the chosen 48 parameters was demonstrated by learning two other classification models using the top 20 and top 10 most important features obtained from the MIMIC explainer (FIG. 5). The performance of the three models on the test set are summarized in Table 3.

TABLE 3
Comparison of Prediction accuracy of models built using
different subsets of parameters.

| | | Prediction Accuracy (test | | | Overall |
|---|---|---|---|---|---|
| | | set) | | | prediction |
| | Training | Positive- | Positive- | | accuracy |
| Parameters | accuracy | hosp. | not hosp. | Negative | (test set) |
| All 48 | 75.6% | 91.7% | 70% | 73.5% | 74% |
| | (335/443) | (11/12) | (35/50) | (36/49) | (82/111) |
| Top 20 | 76.9% | 91.7% | 65% | 72% | 71% |
| | (341/443) | (11/12) | (33/50) | (35/49) | (79/111) |
| Top 10 | 74.9% | 75% | 71.4% | 68% | 70% |
| | (332/443) | (9/12) | (36/50) | (33/49) | (78/111) |

The performance of the top 20-parameter model was observed as inferior to the 48-parameter model in terms of both overall accuracy and sensitivity (or the % positives predicted accurately). The predictive accuracy further declines for the top 10-parameter model, particularly the specificity (or the % negatives predicted accurately). These results show that all 48 parameters are indeed important in distinguishing positives from negatives.

### Use Cases

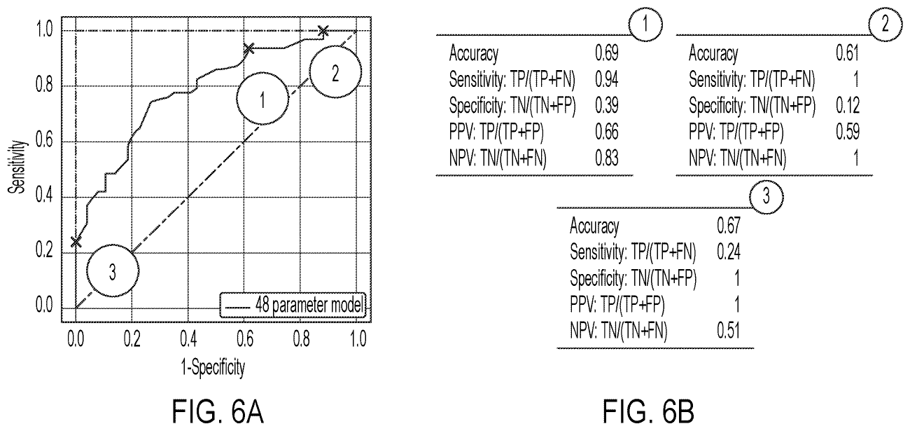

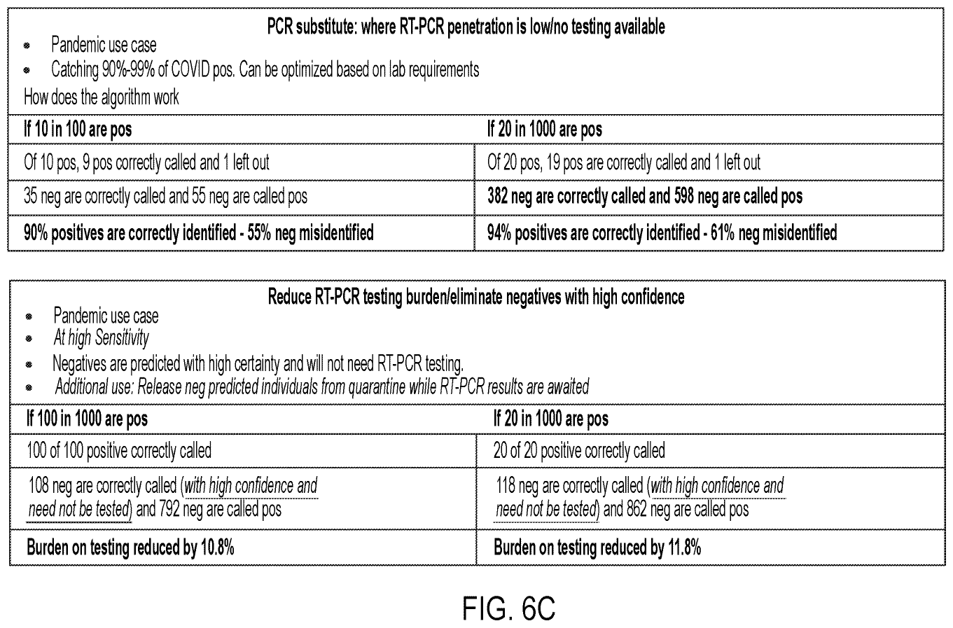

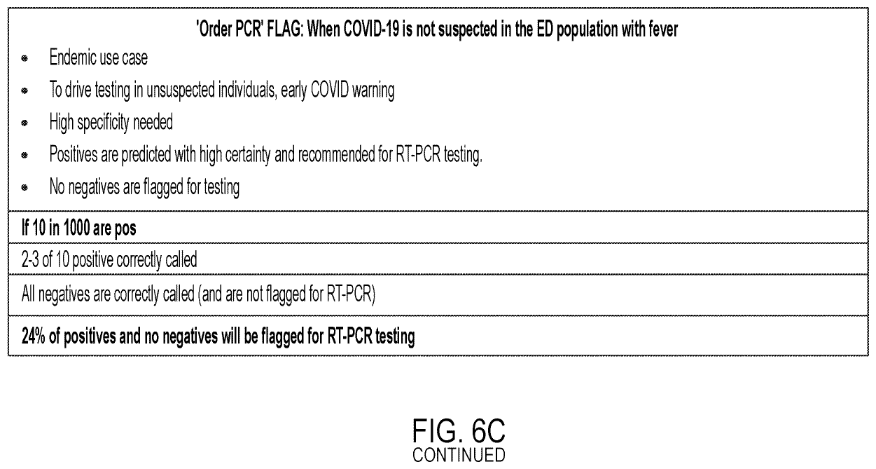

The hematology-based ML algorithm for COVID-19 flagging may be useful in multiple use cases in pandemic and endemic scenarios. In embodiments, the classification threshold may be adjusted to make use of the same prediction model useful in different settings. The present disclosure shows two use cases for a pandemic scenario, and another one for an endemic scenario (FIG. 6A-6C).

In countries facing inaccessibility or scarcity of COVID testing in a pandemic scenario, AI-based COVID-19 flagging using CBC/Diff data may act as a low-cost and rapid alternative. As shown in FIG. 6A, the algorithm may be used at a sensitivity of 0.94 and specificity of 0.39 (point 1 in the ROC curve) to make predictions for this use case. Depending on the sensitivity chosen, the algorithm may capture a large majority of the positives. This result may be used as an additional point in clinical decision making in the absence of a confirmatory test.

In another application in a pandemic scenario, the algorithm may be used at high sensitivity to reduce infection testing burden. This may be especially useful in high volume labs. The rapid turn-around time of CBC/Diff results on the ADVIA 2120i analyzer (about a minute) along with algorithm running time (a few seconds) provides a faster alternative to RT-PCR or antigen testing. At the point 2 in the ROC curve (FIG. 6A), the algorithm has an NPV of 1, where negatives are predicted with high certainty and need not be tested and may be exempted from quarantine. Individuals predicted positive by the algorithm will need to be followed up by COVID specific testing. This approach may reduce testing burden by about 10%.

The algorithm may also be used in an endemic scenario to drive testing in individuals with fever in the emergency department (ED), when COVID-19 is not suspected. The algorithm working at high specificity and positive predictive value (PPV) of 1 (point 3 on the ROC curve) may predict COVID-19 positives, with high certainty, in individuals who would have otherwise been missed, and recommend them for COVID testing. Importantly, at a PPV of 1 no negatives will be flagged for testing, eliminating false alarms.

### DISCUSSION

Embodiments provide a multi-parametric machine learning algorithm for flagging COVID-19 positivity in individuals using parameters obtained from a CBC/Diff test performed on the Siemens Healthineers ADVIA® 2120i Hematology System. A CBC/Diff test is a routinely done blood test and may help detect various health conditions. There are several advantages of coupling the results of a CBC/Diff test with an AI algorithm for flagging infectious diseases. First, all the parameters are acquired on a single instrument, which eliminates the need for aggregating data from multiple sources. Second, such algorithms may act as faster alternatives to standard virus specific testing as the results may be obtained within minutes. Third, machine learning algorithms may help in learning complex relationships between multiple parameters and thus improving the accuracy of diagnosis.

There are several studies that report altered hematology parameters in the presence of COVID-19 infection (See e.g., Henry B M et al., “Hematologic, biochemical and immune biomarker abnormalities associated with severe illness and mortality in coronavirus disease 2019 (COVID-19): a meta-analysis},” Clinical Chemistry and Laboratory Medicine (CCLM) 2020; 58:1021-8; Kermali M et al., “The role of biomarkers in diagnosis of COVID-19—A systematic review,” Life sciences 2020; 254:117788; Lippi G, Plebani M, “Laboratory abnormalities in patients with COVID-2019 infection,” Clinical chemistry and laboratory medicine (CCLM) 2020; 58:1131-4; Wang D et al., “Clinical Characteristics of 138 Hospitalized Patients With 2019 Novel Coronavirus-Infected Pneumonia in Wuhan, China,” JAMA 2020; 323:1061-9; Yip C Y et al., “Temporal changes in immune blood cell parameters in COVID-19 infection and recovery from severe infection,” British Journal of Hematology 2020:33-6; Zhang J-j et al., “Clinical characteristics of 140 patients infected with SARS-CoV-2 in Wuhan, China,” Allergy 2020; 75:1730-41; and Zhang J et al., “Risk factors for disease severity, unimprovement, and mortality in COVID-19 patients in Wuhan, China,” Clinical microbiology and infection 2020; 26:767-72). Another study determines a COVID-19 prognostic score using a linear combination of hemocytometry parameters (See e.g., Linssen J et al., “A novel haemocytometric COVID-19 prognostic score developed and validated in an observational multicentre European hospital-based study,” Elife; 9: e63195).

In contrast, multi-parametric machine learning algorithms may learn complex, non-linear relationships between multiple parameters and improve the overall performance of diagnostic tests.

Embodiments include an ensemble of tree-based algorithms to predict COVID-19 positive or negative status using hematology data. In embodiments, a classifier predicts COVID-19 positivity with an accuracy of 74% on the entire test set and an accuracy of 91.7% on hospitalized patients. Better performance on hospitalized individuals gives excellent confidence in the parameter selection process of the present disclosure. As described in Section 3.4, an algorithm embodiment may advantageously be used in different settings in pandemic and endemic scenarios, including its use as a faster alternative to RT-PCR testing, reducing testing burden in a pandemic and detecting COVID-19 in unsuspected, asymptomatic individuals. Another study (See Kukar M et al., “COVID-19 diagnosis by routine blood tests using machine learning,” Scientific reports 2021; 11:1-9) has used routine blood parameters for COVID-19 diagnosis using a machine learning algorithm, but those parameters need to be collected from multiple different tests, in contrast to embodiments of the present disclosure using parameters obtained from a single instrument. Also, Kukar et al includes a skewed dataset (3% positives, 97% negatives). Though the prevalence rate of COVID-19 at the time of that publication was 3%, it is believed that AUROC is not the right metric for a heavily skewed data set, and perhaps some other metric like the area under the precision-recall curve (AUPRC) may have been a better measure to report cross-validation results. On the other hand, the present disclosure includes a hold-out test dataset (20% of total data) with balanced number of positives and negatives to report AUROC and other metrics.

In embodiments, the algorithm used described herein includes a total of 48 parameters including percentages of various white cell types, like lymphocytes, neutrophils, basophils, monocytes, eosinophils, polymorphonuclear cells, large unstained cells (LUC) and blasts. In embodiments, the parameter set also included platelet count and several red blood cell (RBC) parameters like hemoglobin level, mean corpuscular volume (MCV), cell hemoglobin concentration mean (g/dL), the number of red cells with low, normal and high hemoglobin concentrations and volumes, hemoglobin distribution width (HDW) which is the standard deviation of the hemoglobin concentration (HC) histogram and is expressed in g/dL, red blood cell distribution width (RDW), total RBC counts. In embodiments, the parameter set also included some raw instrument measurements that are unique to the ADVIA 2120i analyzer. In addition to the parameters acquired using ADVIA 2120i, in embodiments, two derived parameters neutrophil-to-lymphocyte ratio (NLR) and LUC-to-lymphocyte ratio were included in the analysis. LUC is a parameter specific to the ADVIA 2120i analyzer.

Some parameters used in an algorithm of the present disclosure have been widely reported to be important in COVID-19 diagnosis and/or prognosis. While eosinopenia has been identified in the literature as an important characteristic of COVID-19 patients (See e.g., Lippi G, Plebani M, “Laboratory abnormalities in patients with COVID-2019 infection,” Clinical chemistry and laboratory medicine (CCLM) 2020; 58:1131-4; and Zhang J-j et al., “Clinical characteristics of 140 patients infected with SARS-COV-2 in Wuhan, China,” Allergy 2020; 75:1730-41), lymphocytes have been observed to be an important biomarker in diagnosis and as well as prognosis of COVID-19 (See e.g., Henry B M et al., “Hematologic, biochemical and immune biomarker abnormalities associated with severe illness and mortality in coronavirus disease 2019 (COVID-19): a meta-analysis},” Clinical Chemistry and Laboratory Medicine (CCLM) 2020; 58:1021-8; Kermali M et al., “The role of biomarkers in diagnosis of COVID-19—A systematic review,” Life sciences 2020; 254:117788; Lippi G, Plebani M, “Laboratory abnormalities in patients with COVID-2019 infection,” Clinical chemistry and laboratory medicine (CCLM) 2020; 58:1131-4; Wang D et al., “Clinical Characteristics of 138 Hospitalized Patients With 2019 Novel Coronavirus-Infected Pneumonia in Wuhan, China,” JAMA 2020; 323:1061-9; Zhang J-j et al., “Clinical characteristics of 140 patients infected with SARS-COV-2 in Wuhan, China,” Allergy 2020; 75:1730-41; Khartabil T et al., “A summary of the diagnostic and prognostic value of hemocytometry markers in COVID-19 patients,” Critical reviews in clinical laboratory sciences 2020; 57:415-31; and Qin C et al., “Dysregulation of immune response in patients with coronavirus 2019 (COVID-19) in Wuhan, China,” Clinical infectious diseases 2020; 71:762-8). Another study (See e.g., Qin C et al., “Dysregulation of immune response in patients with coronavirus 2019 (COVID-19) in Wuhan, China,” Clinical infectious diseases 2020; 71:762-8) reported lower percentages of monocytes, eosinophils, and basophils in severe cases. Leukopenia was observed in 33.7% of hospitalized patients on admission (See e.g., Guan W-j et al., “Clinical characteristics of coronavirus disease 2019 in China,” New England journal of medicine 2020; 382:1708-20). The prevalence of thrombocytopenia has been widely reported in COVID-19 patients, particularly in more severe patients (See e.g., Henry B M et al., “Hematologic, biochemical and immune biomarker abnormalities associated with severe illness and mortality in coronavirus disease 2019 (COVID-19): a meta-analysis},” Clinical Chemistry and Laboratory Medicine (CCLM) 2020; 58:1021-8; Jiang S-Q et al., “The association between severe COVID-19 and low platelet count: evidence from 31 observational studies involving 7613 participants,” British journal of hematology 2020: e29-e33; Kermali M, et al., “The role of biomarkers in diagnosis of COVID-19—A systematic review,” Life sciences 2020; 254:117788; Lippi G, Plebani M, “Laboratory abnormalities in patients with COVID-2019 infection,” Clinical chemistry and laboratory medicine (CCLM) 2020; 58:1131-4; and Lippi G et al., “Thrombocytopenia is associated with severe coronavirus disease 2019 (COVID-19) infections: a meta-analysis,” Clinica chimica acta 2020; 506:145-8). Some studies have also reported the effect of COVID-19 on RBC parameters like hemoglobin, RDW and MCV that are also used in the algorithm of the present disclosure (See e.g., Lippi G, Plebani M, “Laboratory abnormalities in patients with COVID-2019 infection,” Clinical chemistry and laboratory medicine (CCLM) 2020; 58:1131-4; Khartabil T et al., “A summary of the diagnostic and prognostic value of hemocytometry markers in COVID-19 patients,” Critical reviews in clinical laboratory sciences 2020; 57:415-31; and Yu H et al., “Total protein as a biomarker for predicting coronavirus disease-2019 pneumonia,” Preprint with The Lancet 2.

The present disclosure demonstrates embodiments for detecting COVID-19 positivity using hematology parameters derived from a single instrument. This is a specific case study with concurrent COVID-19 positives and negatives from Mumbai, India during the first wave of infection in India. The results are excellent.

This study also highlights the use of a common, non-specific test such as a CBC/Diff for flagging infections and alerting physicians to a potential health condition in a patient. This is possible because of the unique feature of hematology instruments of simultaneously measuring a large number of blood parameters, the values of which may vary depending on the state of health of a patient. In embodiments, such embedded algorithms within the hematology instrument, are useful as an early warning system for the physician and aid in patient management.

### Supplement to Example 1

The training data used here for algorithm development is skewed in terms of gender, containing roughly twice as many males as females. The positive cohort contains 201 males and 108 females, while the negative one has 116 males and 129 females. Of these, the test set consists of 43 males and 19 females in the COVID-19 positive cohort, and 22 males and 27 females in the negative cohort, respectively. However, the model performance is consistent across males and females, with around 74% prediction accuracy for both thus suggesting no sex related bias in prediction (Supplemental Table 1).

TABLE 1
Comparison of prediction accuracy for males and
females in the test data

| | Males | | | Females | | |
|---|---|---|---|---|---|---|
| | Positive- | Positive- | Neg- | Positive- | Positive- | Neg- |
| | hosp. | not hosp. | ative | hosp. | not hosp. | ative |
| #Training | 28 | 130 | 94 | 20 | 69 | 102 |
| examples | | | | | | |
| #Testing | 6 | 37 | 22 | 6 | 13 | 27 |
| examples | | | | | | |
| Accuracy | 100% | 70% | 73% | 83% | 69% | 74% |
| (Correctly | (6/6) | (26/37) | (16/22) | (5/6) | (9/13) | (20/27) |
| predicted/ | | | | | | |
| Total) | | | | | | |

| | |
|---|---|
| 74% | 74% |
| (48/65) | (34/46) |

To know the effect of age on algorithm performance, the prediction accuracy of embodiments of the present disclosure was compared across the test set for different age groups (Supplemental Table 2).

TABLE 2
Comparison of prediction accuracy in the test set
(Correctly predicted/Total) for different age groups

| | Positive- | Positive- | | |
|---|---|---|---|---|
| Age group | hosp. | not hosp. | Negative | Overall |
| 0-20 | — | — | 33% | 33% |
| | | | (1/3) | (1/3) |
| 20-40 | — | 85% | 83% | 84% |
| | | (11/13) | (10/12) | (21/25) |
| 40-60 | 100% | 56% | 83% | 71% |
| | (5/5) | (14/25) | (15/18) | (34/48) |
| 60-80 | 83% | 83% | 64% | 75% |
| | (5/6) | (10/12) | (9/14) | (24/32) |
| 80-90 | 100% | — | 50% | 67% |
| | (1/1) | | (1/2) | (2/3) |

A variation in prediction accuracy was observed across different age groups. This might be due to the small and unequal number of individuals in the test set across different age groups, thus making it difficult to ascertain if age affects algorithm performance. However, the prediction performance of the algorithm for the age group 40-80 (which corresponds to a large majority of the data) is fairly consistent.

The foregoing examples have been provided merely for the purpose of explanation and are in no way to be construed as limiting of the present disclosure. While the disclosure has been described with reference to various embodiments, it is understood that the words, which have been used herein, are words of description and illustration, rather than words of limitation. Further, although the disclosure has been described herein with reference to particular means, materials, and embodiments, the disclosure is not intended to be limited to the particulars disclosed herein; rather, the disclosure extends to all functionally equivalent structures, methods, and uses, such as are within the scope of the appended claims. Those skilled in the art, having the benefit of the teachings of this specification, may effect numerous modifications thereto and changes may be made without departing from the scope and spirit of the disclosure in its aspects.

### Example 2 (Prophetic Example)

Hematology tests such as CBC/Diff (Complete Blood Count with Differential) are ubiquitously performed for overall health monitoring. In embodiments, the present disclosure combines CBC/Diff results from the Siemens Healthineers ADVIA® 2120i Hematology System with a multiparametric machine learning algorithm for rapid flagging of pathogenic disease positivity such as viral, bacterial, or fungal disease positivity. Diagnostic data generation and running the algorithm on the data occur on the same instrument, to provide easy data access. Additionally, the algorithm may also be run on a connected lab information system. Such an algorithm is valuable when access to pathogen testing is limited and for predicting disease state in unsuspected individuals thus prompting a confirmatory test. Various pandemic and endemic applications for this algorithm are demonstrated.

Methods. CBC/Diff data for pathogen positives and negatives are obtained from a preselected cohort. Using data obtained therefrom, machine learning classification models are trained with 5-fold cross-validation for distinguishing positives from negatives.

Results. The final ML model is an ensemble of decision tree-based methods, with excellent accuracy and suitable AUROC on the test set. Significant features include eosinophil and lymphocyte counts are reported earlier. The model is able to predict hospitalized patients more accurately than non-hospitalized patients.

Conclusion. A predictive algorithm, method, and system is provided for rapid pathogenic disease flagging using data from routine CBC testing. The algorithm is useful in pandemic or endemic scenarios as an alternate to testing, reducing testing burden and flagging unsuspected positives.

### Example 3 (Prophetic Example)

Hematology tests such as CBC/Diff (Complete Blood Count with Differential) are ubiquitously performed for overall health monitoring. In embodiments, the present disclosure combines CBC/Diff results from the Siemens Healthineers ADVIA® 2120i Hematology System with a multiparametric machine learning algorithm for rapid flagging of sepsis positivity. Diagnostic data generation and running the algorithm on the data occur on the same instrument, to provide easy data access. Additionally, the algorithm may also be run on a connected lab information system. Such an algorithm is valuable when access to sepsis testing is limited and for predicting sepsis state in unsuspected individuals thus prompting a confirmatory test. Various pandemic and endemic applications for this algorithm are demonstrated.

Methods. CBC/Diff data for pathogen positives and negatives are obtained from a preselected cohort. Using data obtained therefrom, machine learning classification models are trained with 5-fold cross-validation for distinguishing positives from negatives.

Results. The final ML model is an ensemble of decision tree-based methods, with excellent accuracy and suitable AUROC on the test set. Significant features include eosinophil and lymphocyte counts are reported earlier. The model is able to predict hospitalized patients more accurately than non-hospitalized patients.

Conclusion. A predictive algorithm, method, and system is provided for rapid sepsis flagging using data from routine CBC testing. The algorithm is useful in pandemic or endemic scenarios as an alternate to testing, reducing testing burden and flagging unsuspected positives.

Thus, in accordance with the present disclosure, there have been provided systems, articles of manufacture, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

All references cited herein are herein entirely incorporated by reference.

1. A system for predicting a presence of a disease in a subject, the system comprising:
one or more processors;
a medical database coupled to the one or more processors, the medical database comprising patient data; and
a disease prediction module configured to:
receive a plurality of parameters associated with the subject;
determine a threshold for sensitivity and/or specificity associated with a trained machine learning model;
predict, using the trained machine learning model, the presence of the disease in the subject based on a specific threshold of the trained machine learning model and the plurality of parameters associated with the subject; and
output the prediction on an output unit.

2. The system of claim 1, wherein the threshold for sensitivity and/or specificity associated with the trained machine learning model is determined based on a requirement scenario associated with a healthcare provider.

3. The system of claim 2, wherein the requirement scenario associated with the healthcare provider comprises a reduction of infectious disease testing burden or reverse transcriptase polymerase chain reaction (RT-PCR) testing burden, a substitution of infectious disease testing burden, substitution of RT-PCR testing, a determination of a need for testing of subjects, or a combination thereof.

4. The system of claim 1, wherein the plurality of parameters associated with the subject comprises red blood cells (RBC) parameters, platelet parameters, and white blood cells (WBC) parameters.

5. The system of claim 4, wherein the RBC parameters comprise hemoglobin level, hematocrit, RBC size, hemoglobin level in individual RBC, mean corpuscular volume, mean corpuscular hemoglobin concentration, normal RBCs, number of RBCs with hemoglobin concentrations≥28 g/dL and ≤41 g/dL, number of RBCs with hemoglobin volumes≥60 fL and ≤120 fL, hemoglobin distribution width, RBC mean corpuscular volume, RBC volume distribution width, total RBC count, hemoglobin content distribution width, mean of hemoglobin content, or combinations thereof.

6. The system of claim 4, wherein the WBC parameters comprise WBC type, WBC count, WBC percentage, ratio of neutrophils to lymphocytes, ratio of large unstained cells to lymphocytes, number of WBCs indicating pseudobasophilia, WBC maturity parameters, or combinations thereof.

7. The system of claim 5, wherein the WBC parameters further comprise cell hemoglobin concentration mean, number of RBCs with hemoglobin concentrations greater than 41 g/dL, number of RBCs with hemoglobin concentrations less than 28 g/dL, or combinations thereof.

8. The system of claim 1, wherein the trained machine learning model is preselected.

9. The system of claim 1, wherein the disease prediction module is further configured to, using the trained machine learning model, predict a need for stratification/severity or hospitalization of the subject based on the plurality of the parameters associated with the subject.

10. The system of claim 1, wherein the disease prediction module is further configured to predict occurrence of long COVID-19 in the subject.

11. A method of predicting a presence of a disease in a subject, the method comprising:
receiving a plurality of parameters associated with the subject;
determining a threshold for sensitivity and/or specificity associated with a trained machine learning model;
predicting, using the trained machine learning model, the presence of the disease in the subject based on a specific threshold of the trained machine learning model and the plurality of parameters associated with the subject; and
outputting the prediction on an output unit.

12. The method of claim 11, wherein the threshold for sensitivity and/or specificity associated with the trained machine learning model is determined based on a requirement scenario associated with a healthcare provider.

13. The method of claim 12, wherein the requirement scenario associated with the healthcare provider comprises a reduction of reverse transcriptase polymerase chain reaction (RT-PCR) or other infectious disease testing burden, a substitution of such testing, a determination of a need for testing of subjects, or a combination thereof.

14. The method of claim 11, wherein the disease is COVID-19.

15. The method of claim 11, further comprising:
predicting a need for hospitalization of the subject based on the plurality of the parameters associated with the subject, using the trained machine learning model; and
predicting an occurrence of long COVID-19 in the subject.

16. A system or a component of the system comprising a non-transitory computer-readable medium with instructions encoded thereon, the instructions configured to cause one or more processors to:
receive a plurality of parameters associated with a subject;
determine a threshold for sensitivity and/or specificity associated with a trained machine learning model;
predict, using the trained machine learning model, a presence of a disease in the subject based on a specific threshold of the trained machine learning model and the plurality of parameters associated with the subject; and
output the prediction on an output unit.

17. (canceled)
